(19) United States
(12) Patent Application Publication
(10) Pub. No.: US 2017/0088496 A1
(43) Pub. Date: Mar. 30, 2017

(54) PROCESSES AND SYSTEMS FOR GENERATING GLYCEROL ETHERS THROUGH TRANSETHERIFICATION

(71) Applicant: SAUDI BASIC INDUSTRIES CORPORATION, Riyadh (SA)

(72) Inventors: Luis Aramburo, Maastricht (NL); Christoph Dittrich, Aachen (DE); Antonio Matarredona, Maastricht (NL)

(21) Appl. No.: 15/126,710

(22) PCT Filed: Mar. 17, 2015
(86) PCT No.: PCT/IB2015/051952

§ 371 Date: Sep. 16, 2016

Related U.S. Application Data
(60) Provisional application No. 61/954,686, filed on Mar. 18, 2014.

(51) Int. Cl. C07C 41/14; C07C 41/54; C10L 1/02; C07C 41/06; C07C 7/10; B01J 19/24; C07C 41/60; C07C 41/50

(52) U.S. Cl. CPC C07C 41/14; C07C 41/60; C07C 41/54; C07C 41/50; C07C 41/06; C07C 7/10; B01J 19/245; C10L 1/026; B01J 2219/24; C10L 2200/0476

(57) ABSTRACT

A process of generating a glycerol ether is provided. The process includes reacting isobutylene with an alcohol to obtain a tertiary alkyl ether through an etherification reaction and generating a glycerol ether from the tertiary alkyl ether and glycerol through a transetherification reaction, A system for generating a glycerol ether is also provided.

### TECHNICAL FIELD

The presently disclosed subject matter relates to processes and systems for generating glycerol ethers.

### BACKGROUND

Glycerol, also known as glycerin, is a colorless, odorless, viscous liquid. Glycerol has various uses, including, for example, as an antifreeze agent and as an excipient in certain pharmaceutical preparations. Glycerol occurs in nature and can also be prepared synthetically by various routes. Among the sources of glycerol is commercial production of biodiesel. Glycerol can be a waste product in the generation of biodiesel.

Biodiesel is a fuel or component of fuel that can be used for various purposes, including powering diesel motors. Biodiesel has received attention as a renewable fuel and can be a complement to and/or substitute for various fossil fuels. Biodiesel is typically a mixture of chemical compounds, including alkyl esters of natural fatty acids, and can be generated through the transesterification of triglycerides with simple alcohols in the presence of a catalyst. Such transesterification reactions generate alkyl esters of fatty acids along with glycerol as a byproduct.

Biodiesel fuels can be mixed with various additives, including glycerol ethers. Glycerol ethers are ether compounds wherein at least one of the carbon moieties attached to an ether linkage is derived from glycerol. Glycerol ethers can be derived from renewable sources and are accordingly of interest as a renewable source of energy. Glycerol ethers can be added to gasoline, diesel, biodiesel, and other fuels and can impart various desirable properties to the fuel. For example, glycerol ethers can be added to fuels as an oxygenate to improve the performance of the fuels. Glycerol ethers can be soluble in various fuels, including biodiesel, and can be otherwise compatible with the fuels.

As glycerol is a triol and has three hydroxyl groups capable of derivatization into ether linkages, glycerol ethers include mono-, di-, and triether compounds. Examples of glycerol ethers include glycerol tert-butyl ethers (GTBEs). Five structurally distinct glycerol tert-butyl ethers can be formed: (1) 1-tert-butyl glycerol (3-(tert-butoxy)propane-1,2-diol); (2) 2-tert-butyl glycerol (2-(tert-butoxy)propane-1,3-diol); (3) 1,3-di-tert-butyl glycerol (1,3-di-(tert-butoxy)propan-2-ol); (4) 1,2-di-tert-butyl glycerol (1,2-di-(tert-butoxy)propan-3-ol); and (5) tri-tert-butyl glycerol (1,2,3-tri-(tert-butoxy)propane). The first two compounds, 1-tert-butyl glycerol and 2-tert-butyl glycerol, are mono-tert-butyl glycerol ethers (mono-GTBEs, also known as m-GTBEs). The next two compounds, 1,3-di-tert-butyl glycerol and 1,2-di-tert-butyl glycerol, are di-tert-butyl glycerol ethers (di-GTBEs). The last compound, tri-tert-butyl glycerol, is tri-tert-butyl glycerol ether (tri-GTBE, also known as t-GTBE). Di-GTBEs and tri-GTBEs are sometimes known together as higher GTBEs, or h-GTBEs.

GTBEs can have useful properties as fuel additives, particularly as compared to certain existing additives. For example, methyl tert-butyl ether (MTBE) is soluble in diesel, biodiesel, and other fuels and is a commonly used oxygenate fuel additive. However, MTBE has relatively high water solubility and can cause hazardous water contamination. Like MTBE, GTBEs can be soluble in diesel, biodiesel, and other fuels and can be used as oxygenate fuel additives. In particular, di-GTBEs and tri-GTBE are desirable as fuel additives, as they have good solubility in diesel and biodiesel fuels. However, unlike MTBE, di-GTBEs and tri-GTBE have low solubility in water, which makes them less likely to cause water contamination.

Glycerol ethers can also be appealing because they can be generated from glycerol, which, as noted above, is often a byproduct of biodiesel production. Glycerol ether formation can accordingly convert a relatively low value compound (glycerol) into a useful product (glycerol ethers).

Certain existing processes of generating glycerol ethers can involve transetherification reactions of glycerol and an ether. For example, International Publication No. WO 2010/053354 A2 to Groeneveld et al. (Groeneveld) briefly describes processes of generating GTBEs and glycerol tert-amyl ethers (GTAEs) through transetherification reactions of glycerol with MTBE and glycerol with methyl tert-amyl ether (MTAE), respectively. Groeneveld describes preferential generation of m-GTBEs over di- and tri-GTBEs. Groeneveld does not disclose any processes of preparing tertiary alkyl ethers (e.g., MTBE or MTAE), nor does it disclose any processes of preparing glycerol ethers that permit the conversion of abundant, economical feedstock compounds including isobutylene and alcohols into valuable glycerol ethers.

Other existing processes of generating glycerol ethers can involve etherification reactions of glycerol and an alkene. For example, glycerol can be reacted with isobutylene to form GTBEs. The reaction of glycerol with isobutylene can suffer from various drawbacks. Etherification of glycerol with isobutylene can suffer from mass transfer limitations caused by a non-optimal contact between isobutylene and glycerol liquid phases. Other drawbacks with etherification of glycerol with isobutylene can include undesired secondary reactions (e.g., oligomerization of isobutylene), poor control of product selectivity (e.g., poor control of the relative output of m-GTBEs, di-GTBEs, and tri-GTBE), and limited catalyst lifetime. Moreover, high purity glycerol can be required to perform the etherification of glycerol with isobutylene to generate GTBEs. High purity glycerol can be expensive.

Thus, there remains a need in the art for improved processes and systems for generating glycerol ethers with improved properties, including improved economy, efficiency, and selectivity.

### SUMMARY OF THE DISCLOSED SUBJECT MATTER

The presently disclosed subject matter provides processes and systems for generating a glycerol ether.

In one embodiment, a non-limiting exemplary process of generating a glycerol ether includes reacting isobutylene with an alcohol to obtain a tertiary alkyl ether through an etherification reaction and generating a glycerol ether from the tertiary alkyl ether and glycerol through a transetherification reaction.

In one embodiment of the presently disclosed subject matter, a non-limiting exemplary system for generating a glycerol ether includes a first section for generating a tertiary alkyl ether. The first section includes at least one tertiary alkyl ether reactor coupled to an isobutylene feed line and an alcohol feed line. The first section further includes at least one separation unit coupled to the tertiary alkyl ether reactor. The first section further includes a tertiary alkyl ether outlet line coupled to the separation unit. The first section also includes a tertiary alkyl ether storage tank coupled to the tertiary alkyl ether outlet line and configured to store a tertiary alkyl ether. The non-limiting exemplary system further includes a second section for generating a glycerol ether from a tertiary alkyl ether and glycerol. The second section includes a glycerol ether production unit coupled to a tertiary alkyl ether feed line. The tertiary alkyl ether feed line is further coupled to the tertiary alkyl ether storage tank of the first section. The glycerol ether production unit is also coupled to a glycerol feed line. The second section also includes a glycerol ether product line configured to remove a glycerol ether from the glycerol ether production unit.

In certain embodiments of the presently disclosed subject matter, the system for generating a glycerol ether can include an alcohol storage tank coupled to the alcohol feed line.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram depicting an exemplary system for generating a glycerol ether in accordance with one non-limiting embodiment of the disclosed subject matter.

FIG. 2 is a schematic diagram depicting another exemplary system for generating a glycerol ether in accordance with one non-limiting exemplary embodiment of the disclosed subject matter.

FIG. 3 is a series of chemical equations depicting certain non-limiting examples of disproportionation and/or decomposition reactions that glycerol ether compounds can undergo.

### DETAILED DESCRIPTION

The presently disclosed subject matter provides processes and systems for generating glycerol ethers.

In one embodiment, a non-limiting exemplary process includes reacting isobutylene with an alcohol to obtain a tertiary alkyl ether through an etherification reaction and generating a glycerol ether from the tertiary alkyl ether and glycerol through a transetherification reaction. The glycerol ether can include a glycerol tert-butyl ether (GTBE). The glycerol tert-butyl ether (GTBE) can include one or more of the ethers selected from di-tert-butyl glycerol ethers (di-GTBEs) and tri-tert-butyl glycerol ether (tri-GTBE). In certain embodiments, the process includes generating a mixture of mono-tert-butyl glycerol ethers (m-GTBEs), di-GTBEs, and tri-GTBE. In certain embodiments of the presently disclosed subject matter, the tertiary alkyl ether can be methyl tert-butyl ether. In certain embodiments, the alcohol can be methanol. In certain embodiments, molecular sieves can be used in the transetherification reaction.

In certain embodiments, the process of generating a glycerol ether can further include obtaining isobutylene from a C₄ hydrocarbon mixture of alkanes and alkenes. The C₄ hydrocarbon mixture of alkanes and alkenes can be C₄ raffmate-1. The process can further include obtaining the C₄ hydrocarbon mixture of alkanes and alkenes from extracting butadiene from a C₄ hydrocarbon mixture.

In certain embodiments of the presently disclosed subject matter, the transetherification reaction can be catalyzed by one or more catalysts selected from the group consisting of liquid acids and solid acids. One or more of the catalysts can be an ion-exchange resin.

In certain embodiments of the presently disclosed subject matter, the transetherification reaction can regenerate an alcohol. The process of generating a glycerol ether can further include feeding the alcohol regenerated in the transetherification reaction into the etherification reaction.

In certain embodiments of the presently disclosed subject matter, the process of generating a glycerol ether can further include obtaining glycerol from a biodiesel process. The biodiesel process can include reacting fatty acids with an alcohol to generate biodiesel and glycerol. By way of non-limiting example, the alcohol can be methanol.

In certain embodiments, the process of generating a glycerol ether can further include feeding an alcohol generated in the transetherification reaction into a biodiesel process. The biodiesel process can include reacting fatty acids with the alcohol to generate biodiesel and glycerol. By way of non-limiting example, the alcohol can be methanol. The process can further include feeding the glycerol generated by the biodiesel process into the transetherification reaction.

In certain embodiments, the generation of glycerol ethers can occur in a single liquid phase. Generating a glycerol ether through transetherification in a single liquid phase can have advantages over other methods of generating a glycerol ether, including improved mass transfer, improved reaction rate, and improved control of product selectivity. Certain existing processes and systems for generating glycerol ethers typically involve etherification reactions of glycerol and an alkene (e.g., isobutylene), which are catalyzed by a catalyst and which are generally characterized by two distinct, immiscible liquid phases: a relatively polar phase rich in glycerol and a relatively non-polar phase rich in alkene. The existence of two distinct, immiscible liquid phases implies non-intimate contact between the phases. Non-intimate contact between the phases can create mass transfer limitations, which can reduce the reaction rate and can accelerate undesired side reactions. Examples of undesired side reactions can include oligomerization of the alkene, disproportionation reactions of the glycerol ether products, and decomposition reactions of the glycerol ether products.

FIG. 3 depicts certain non-limiting examples of disproportionation and/or decomposition side reactions that mono-, di-, and tri-GTBE compounds can undergo. Reduced reaction rate can increase the required residence time of the reaction, which can in turn increase costs associated with the process. Undesired side reactions reduce the yield of desired products, i.e., glycerol ethers, and can also reduce catalyst activity and lifetime, as products of the side reactions (e.g., oligomerized alkenes) can reversibly and/or irreversibly bind to the catalyst.

In certain embodiments of the presently disclosed subject matter, the transetherification reaction of a tertiary alkyl ether and glycerol to generate a glycerol ether can occur with the tertiary alkyl ether in a gas phase. For example, in a non-limiting embodiment, the tertiary alkyl ether can be MTBE, MTBE can be used in the gas phase, and the catalyst can be a heterogeneous catalyst.

In certain embodiments of the presently disclosed subject matter, the tertiary alkyl ether can be methyl tert-butyl ether (MTBE). In certain embodiments, the tertiary alkyl ether can be ethyl tert-butyl ether (ETBE). In certain embodiments, the alcohol can be a simple, abundant, economical alkyl alcohol, e.g., methanol, ethanol, or 1-propanol. In certain embodiments, the alcohol can be methanol. In certain embodiments, the alcohol can be ethanol.

In certain embodiments, molecular sieves can be used in the transetherification reaction. For example, molecular sieves can be added to a glycerol ether production unit 109, 209 that includes a reactor where a transetherification reaction is performed. Molecular sieves can improve the transetherification reaction. The molecular sieves can be of the sort known to one to ordinary skill in the art, e.g., 4A molecular sieves. Without being bound to any particular theory, it can be that molecular sieves can act as a selective methanol trap. In this manner, transetherification may be improved with use of molecular sieves by removal of methanol from the reaction mixture, enhancing the rate of reaction of MTBE. Moreover, it can be that molecular sieves can also improve the reaction by removing and/or trapping water that may be present in the reaction mixture. Water can degrade the performance of a transetherification reaction, both by promoting the permanent loss of active sites on a transetherification catalyst (e.g., by sulfonation) and by competing for active sites on the transetherification catalyst (e.g., by reversible adsorption). Reducing water content in a transetherification reaction can improve performance by increasing reaction stability and catalyst life. Reducing water content can also improve product selectivity.

The glycerol used in the transetherification reaction can be high purity glycerol, but the processes and systems for generating glycerol ethers of the presently disclosed subject matter do not require the use of high purity glycerol. For example, the transetherification reaction of the presently disclosed subject matter can tolerate glycerol that is contaminated with some amount of methanol and/or water. As noted above, molecular sieves can be used in the transetherification reaction, and the molecular sieves can serve to remove methanol and water from the reaction mixture. Moreover, the catalysts used in the transetherification reaction of the presently disclosed subject matter can be tolerant of some amount of methanol and/or water. Accordingly, the processes and systems for generating glycerol ethers of the presently disclosed subject matter can permit use of lower purity (and lower cost) glycerol than existing processes and systems, which involve etherification reactions of glycerol and an alkene and can require high purity glycerol.

In certain embodiments, the transetherification may involve various ratios of the tertiary alkyl ether and glycerol. For example, in certain non-limiting embodiments, the process of generating a glycerol ether can include a transetherification reaction of MTBE and glycerol in which the molar ratio of MTBE to glycerol can be about 3:1 or greater than about 3:1. The relative concentration of the tertiary alkyl ether with respect to glycerol can be augmented without inducing a significant increase in the rate of undesired side reactions. Use of a high ratio of tertiary alkyl ether with respect to glycerol can translate into a faster and more effective process of generating a glycerol ether. By contrast, existing methods for generating a glycerol ether that involve an etherification reaction of an alkene and glycerol typically suffer from increased side reactions (e.g., oligomerization of the alkene) when high ratios of alkene with respect to glycerol are used.

In certain embodiments, the process of generating a glycerol ether can further include obtaining isobutylene from a C₄ hydrocarbon mixture of alkanes and alkenes. The C₄ hydrocarbon mixture of alkanes and alkenes can be C₄ raffinate-1. The process can further include obtaining the C₄ hydrocarbon mixture of alkanes and alkenes from extracting butadiene from a C₄ hydrocarbon mixture that contains butadiene. For example, C₄ raffinate-1 can be obtained from extracting butadiene from a C₄ hydrocarbon mixture that contains butadiene, e.g., a C4 hydrocarbon mixture containing butadiene that can be derived from naphtha steam crackers and other petrochemical plants. Obtaining isobutylene from a C₄ hydrocarbon mixture of alkanes and alkenes, e.g., C₄ raffinate-1, and reacting isobutylene with an alcohol to obtain a tertiary alkyl ether can improve the overall efficiency of the process. For example, in certain non-limiting embodiments, isobutylene can be obtained from a C₄ hydrocarbon mixture of alkanes and alkenes, e.g., C₄ raffinate-1, and reacted with methanol to obtain MTBE. When isobutylene is obtained from a C₄ hydrocarbon mixture of alkanes and alkenes, e.g., C₄ raffinate-1, other components present in the C₄ hydrocarbon mixture of alkanes and alkenes can be removed. When isobutylene is obtained from a C₄ hydrocarbon mixture of alkanes and alkenes, e.g., C₄ raffinate-1, and reacted with methanol to obtain MTBE, only a limited amount of tert-butyl alcohol (TBA) is allowed into the transetherification process. TBA can be present in the C₄ hydrocarbon mixture of alkanes and alkenes and/or produced from an undesired reaction of water and isobutylene. Reducing the amount of TBA allowed into the transetherification process can be beneficial, as TBA can degrade catalyst activity and lifetime. Also, when isobutylene is obtained from a C₄ hydrocarbon mixture of alkanes and alkenes, e.g., C₄ raffinate-1, and reacted with methanol to obtain MTBE, very low levels of other impurities (e.g., traces of solvent from butadiene extraction, metals, and salts) can be present in the MTBE as compared to MTBE obtained from other sources. The MTBE obtained from reaction of isobutylene can then be reacted with glycerol in a transetherification reaction to generate a glycerol ether. The transetherification reaction can be performed in a glycerol ether production unit 109, 209 that includes a reactor wherein MTBE and glycerol are mixed. As compared to etherification reactions of isobutylene and glycerol in which a C₄ hydrocarbon mixture of alkanes and alkenes, e.g., C₄ raffinate-1, is used directly, the process of obtaining isobutylene from a C₄ hydrocarbon mixture of alkanes and alkenes, reacting the obtained isobutylene with methanol to obtain MTBE, and reacting the obtained MTBE with glycerol in a transetherification reaction has numerous advantages, which include decreased reaction volume in the reactor of the glycerol ether production unit 109, 209, decreased residence time required to arrive at a specific glycerol conversion, and reduced quantities of compounds that can poison the reaction catalyst.

In certain embodiments of the presently disclosed subject matter, the transetherification reaction can be catalyzed by one or more catalysts selected from liquid acids and solid acids. One or more of the catalysts can be an ion-exchange resin. The ion-exchange resin can be an acidic ion-exchange resin, i.e., the ion-exchange resin can be a solid acid catalyst. Suitable acid catalysts can include Bronsted acids and Lewis acids. In certain embodiments, the transetherification may be catalyzed by homogenous catalysts. In other embodiments, the transetherification may be catalyzed by heterogeneous catalysts, or a combination of homogenous and heterogeneous catalysts. By way of non-limiting example, suitable catalysts for the transetherification reaction can include sulfuric acid, acetic acid, formic acid, hydrochloric acid, sulfamic acid, methanesulfonic acid, phosphoric acid, trifluoroacetic acid, thionyl chloride, Amberlyst™ resins, Amberlite™ resins, and other catalysts known to one of ordinary skill in the art to be capable of catalyzing transetherification reactions. Compared with existing processes of generating glycerol ethers that involve a direct etherification reaction between an alkene and glycerol, the transetherification reaction of a tertiary alkyl ether and glycerol can be performed under milder conditions. Milder conditions can enable lower reaction temperatures, reduced side reactions, and better control over product selectivity. The lower hydrophobicity of tertiary alkyl ethers as compared to alkenes can enable improved wettability of the catalyst and improved contact between reactants in the transetherification reactions of the presently disclosed subject matter as compared to existing processes of generating glycerol ethers.

As noted above, ion-exchange resins can be used in the transetherification reaction of the presently disclosed subject matter as a catalyst for the reaction. In certain embodiments, ion-exchange resins can also be used in the presence of another catalyst to improve the transetherification reaction. For example, ion-exchange resins can be used to purify various reactants and/or the reaction mixture, e.g., by removing particular ions.

The presently disclosed subject matter can include processes of generating glycerol ethers from a tertiary alkyl ether and glycerol through a transetherification reaction, wherein the transetherification reaction is characterized by improved contact between reactants, improved reaction selectivity, and improved catalyst lifetime. These improvements can derive from the presence of a single reaction phase, a decrease in undesired side reactions, and a reduction in the amount of poisons reaching the transetherification reactor, among other reasons. For example, the presently disclosed subject matter can include processes of generating GTBEs from MTBE and glycerol through a transetherification reaction, wherein certain products, e.g., di-GTBEs and/or tri-GTBEs, are generated selectively over other products, e.g., m-GTBEs.

In certain embodiments of the presently disclosed subject matter, the transetherification reaction can regenerate an alcohol. The process of generating a glycerol ether can further include feeding the alcohol regenerated in the transetherification reaction into the etherification reaction.

In certain embodiments of the presently disclosed subject matter, the process of generating a glycerol ether can further include obtaining glycerol from a biodiesel process. The biodiesel process can include reacting fatty acids with an alcohol to generate biodiesel and glycerol. The biodiesel process can include a transesterification reaction. By way of non-limiting example, the alcohol can be methanol.

In certain embodiments, the process of generating a glycerol ether can further include feeding an alcohol generated in the transetherification reaction into a biodiesel process. The biodiesel process can include reacting fatty acids with the alcohol to generate biodiesel and glycerol. The biodiesel process can include a transesterification reaction. By way of non-limiting example, the alcohol can be methanol. The process can further include feeding the glycerol generated by the biodiesel process into the transetherification reaction.

The transetherification reaction of the presently disclosed subject matter can be performed and optimized in various ways known in the art. By way of non-limiting example, the transetherification reaction of a tertiary alkyl ether and glycerol can be performed at a temperature in a range from about 20° C. to about 200° C. In certain embodiments, the reaction can be performed at a temperature in a range from about 50° C. to about 100° C. By way of non-limiting example, the transetherification reaction of a tertiary alkyl ether and glycerol can be performed with a molar ratio of tertiary alkyl ether to glycerol of about 1:1 to about 20:1. In certain embodiments, the reaction can be performed with a molar ratio of tertiary alkyl ether to glycerol of about 1:1 to about 9:1. By way of non-limiting example, the transetherification reaction of a tertiary alkyl ether and glycerol can be performed with a catalyst loading of about 0.01% to about 25% wt %, as compared to the weight of glycerol. In certain embodiments, the reaction can be performed with catalyst loading of about 3% to about 7.5% wt %, as compared to the weight of glycerol. By way of non-limiting example, the transetherification reaction of a tertiary alkyl ether and glycerol can be performed at a pressure in of about 0.1 MegaPascals (MPa) to about 5.0 MPa (about 1 bar to about 50 bar). In certain embodiments, the reaction can be performed at a pressure of about 1.2 MPa to about 1.8 MPa (about 12 bar to about 18 bar). By way of non-limiting example, the transetherification reaction of a tertiary alkyl ether and glycerol can be performed at a stirring speed of about 10 revolutions per minute (rpm) to about 2500 rpm. In certain embodiments, the reaction can be performed at a stirring speed of about 1200 rpm.

In one embodiment of the presently disclosed subject matter, a non-limiting exemplary system for generating a glycerol ether includes a first section for generating a tertiary alkyl ether. The first section includes at least one tertiary alkyl ether reactor coupled to an isobutylene feed line and an alcohol feed line. The first section further includes at least one separation unit coupled to the tertiary alkyl ether reactor. The first section further includes a tertiary alkyl ether outlet line coupled to the separation unit. The first section also includes a tertiary alkyl ether storage tank coupled to the tertiary alkyl ether outlet line and configured to store a tertiary alkyl ether. The non-limiting exemplary system further includes a second section for generating a glycerol ether from a tertiary alkyl ether and glycerol. The second section includes a glycerol ether production unit coupled to a tertiary alkyl ether feed line. The tertiary alkyl ether feed line is further coupled to the tertiary alkyl ether storage tank of the first section. The glycerol ether production unit is also coupled to a glycerol feed line. The second section also includes a glycerol ether product line configured to remove a glycerol ether from the glycerol ether production unit.

In certain embodiments of the presently disclosed subject matter, the system for generating a glycerol ether can further include an alcohol outlet line coupled to the glycerol ether production unit of the second section. The alcohol outline line can be configured to remove an alcohol from the glycerol ether production unit. The system can further include a third section for generating biodiesel. The third section can include a biodiesel production unit coupled to a fatty acid feed line and the alcohol outlet line of the second section. The third section can also include a biodiesel product line configured to remove biodiesel from the biodiesel production unit. The second section and third section can be configured so that the glycerol feed line coupled to the glycerol ether production unit of the second section is further coupled to the biodiesel production unit of the third section, such that glycerol generated in the biodiesel production unit can be removed to the glycerol ether production unit.

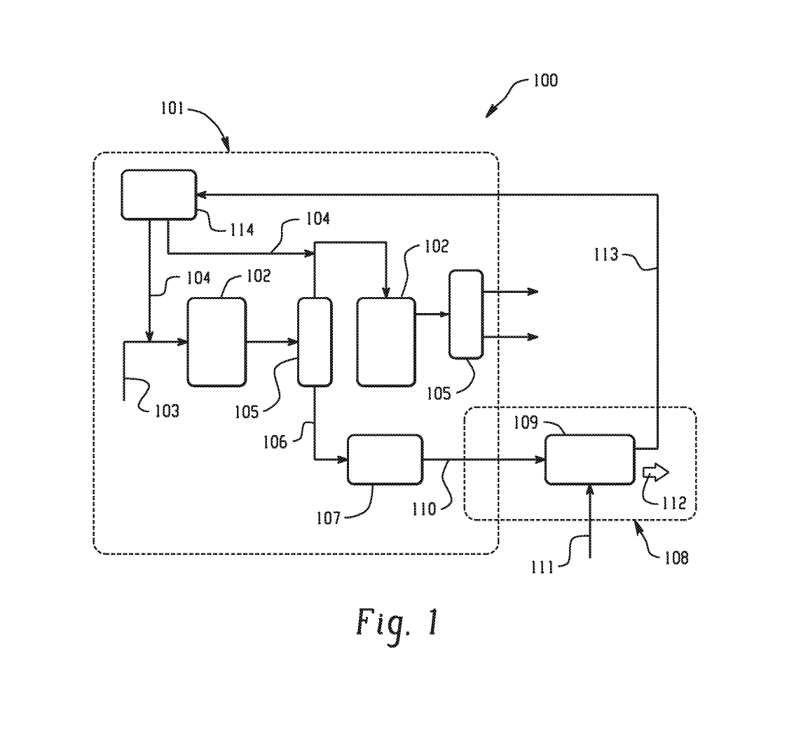

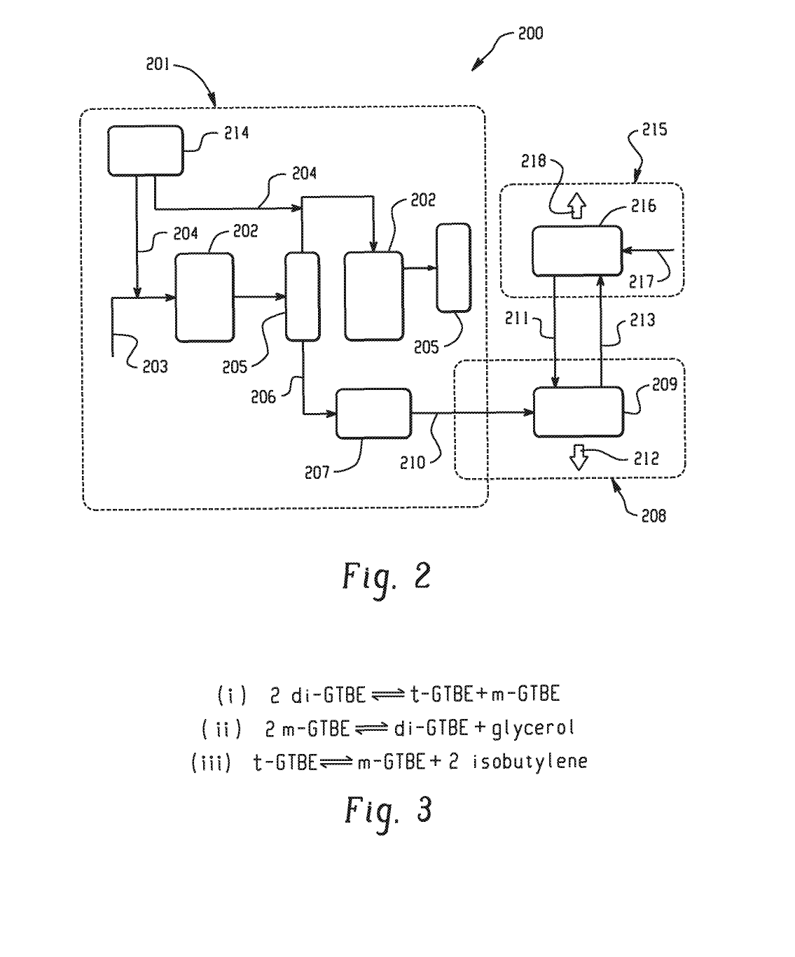

For the purpose of illustration and not limitation, FIGS. 1 and 2 are schematic representations of an exemplary system for generating glycerol ethers according to the disclosed subject matter. The system 100, 200 can include a first section 101, 201 for generating a tertiary alkyl ether. In certain non-limiting embodiments, the first section 101, 201 can be a system for generating a tertiary alkyl ether that uses existing technology. For example, the first section 101, 201 can be an existing facility for production of MTBE. Use of an existing production facility for production of MTBE can have a positive impact on the overall economics of the process of generating glycerol ethers.

The first section 101, 201 can include at least one tertiary alkyl ether reactor 102, 202 coupled to an isobutylene feed line 103, 203 and an alcohol feed line 104, 204. In certain embodiments, the system 100, 200 and the first section 101, 201 can include an alcohol storage tank 114, 214, which can be coupled to the alcohol feed line 104, 204. The first section 101, 201 can further include at least one separation unit 105, 205, which can be coupled to the tertiary alkyl ether reactor. The separation unit 105, 205 can be a distillation column. The separation unit 105, 205 can separate one or more tertiary alkyl ethers from other components. The first section 101, 201 can also include a tertiary alkyl ether outlet line 106, 206 coupled to the separation unit 105, 205. The tertiary alkyl ether outline line 106, 206 can be coupled to a tertiary alkyl ether storage tank 107, 207, which can be configured to store a tertiary alkyl ether.

The system 100, 200 can further include a second section 108, 208 for generating a glycerol ether from a tertiary alkyl ether and glycerol. The system 100, 200 can be configured so that the first section 101, 201 and the second section 108, 208 can be operated flexibly and/or simultaneously.

The second section 108, 208 can include a glycerol ether production unit 109, 209, which can be coupled to a tertiary alkyl ether feed line 110, 210. The glycerol ether production unit 109, 209 can include a reactor in which glycerol ethers are generated, e.g., through transetherification reactions, and can farther include one or more separation units that can separate glycerol ethers from other components. For example, one or more separation units in the glycerol ether production unit 109, 209 can, in certain non-limiting embodiments, separate di- and tri-GTBEs from mono-GTBEs and glycerol. The tertiary alkyl ether feed line 110, 210 can be further coupled to the tertiary alkyl ether storage tank 107, 207 of the first section. The glycerol ether production unit 109, 209 can be further coupled to a glycerol feed line 111, 211. The glycerol ether production unit 109,209 can be further coupled to a glycerol ether product line 112, 212, which can be configured to remove a glycerol ether from the glycerol ether production unit 109, 209.

In certain embodiments of the presently disclosed subject matter, the system 200 for generating a glycerol ether can further include an alcohol outline line 213 coupled to the glycerol ether production unit 209 of the second section 208. The alcohol outline line 213 can be configured to remove an alcohol from the glycerol ether production unit 209. The system 200 can further include a third section 215 for generating biodiesel. The third section can include a biodiesel production unit 216, which can be coupled to a fatty acid feed line 217 and the alcohol outline line 213 of the second section. The biodiesel production unit 216 can include a reactor in which biodiesel is generated and can further include one or more separation units that can separate biodiesel from other components. The third section 215 can further include a biodiesel product line 218 configured to remove biodiesel from the biodiesel production unit. The second section 208 and third section 215 can be configured so that the glycerol feed line 211 coupled to the glycerol ether production unit 209 of the second section is further coupled to the biodiesel production unit 216 of the third section 215, such that glycerol generated in the biodiesel production unit 216 can be removed to the glycerol ether production unit 209.

The system 100, 200 of the presently disclosed subject matter can be operated continuous, semi-continuous, or batch mode. The various sections of the system 100, 200 can be operated simultaneously or, alternatively, can be operated separately.

The reactors can be constructed of any suitable materials such as, but not limited to, metals, alloys including steel, glass, enamels, ceramics, polymers, plastics, and combinations comprising at least one of the foregoing. The reactors can include reaction vessels and reaction chambers of any suitable design and shape such as, but not limited to, tubular, cylindrical, rectangular, dome, or bell shaped. The dimensions and size of the reactors can vary depending on the desired reaction type, production capacity, feed type, and catalyst. For example, the reactor size can be about 50 milliliters (mL) (e.g., for lab reactors) to about 20,000 liters (L) (e.g., for commercial reactors). The geometry of the reactors can be adjustable in various ways known to one of ordinary skill in the art.

The processes and systems of the presently disclosed subject matter can have various advantages over existing processes and systems for generating glycerol ethers. For example, in certain embodiments of the presently disclosed subject matter, GTBE is generated. In certain embodiments, the production of GTBE through a transetherification reaction of a tertiary alkyl ether and glycerol can be coupled with the generation of biodiesel from fatty acids. In certain embodiments, the tertiary alkyl ether can be MTBE. In these embodiments, methanol is produced as a byproduct of the transetherification reaction in the glycerol ether production unit 109, 209. The methanol produced as a byproduct of the transetherification reaction can be removed from the glycerol ether production unit 109, 209 through an alcohol outlet line 113, 213. In certain embodiments, the alcohol outlet line 213 can feed methanol to the biodiesel production unit 216, where the methanol can react with fatty acids to generate biodiesel and glycerol. The glycerol produced as a byproduct of the biodiesel reaction can be removed from the biodiesel production unit 216 through a glycerol feed line 211, which can feed the glycerol to the glycerol ether production unit 209. In the glycerol ether production unit 209, the glycerol can react with MTBE through a transetherification reaction to generate glycerol ethers.

Thus the presently disclosed subject matter provides processes and systems in which various byproducts of certain reactions and reactors are not wasted but are instead recycled in other reactions and reactors. These processes and systems can have advantages over certain existing processes and systems for generating glycerol ethers, which can include the following: more efficient and safer transport and storage of the tertiary alkyl ether (e.g., MTBE) and other reaction components; the absence of generating water, which can improve catalyst activity and lifetime; reduced waste generation; and more efficient design and operation of reactors, as each reactor can be optimized and operated individually. Other advantages can include the use of abundant, economical feedstock compounds including isobutylene and alcohols (e.g., methanol or ethanol) as starting materials, which can improve the overall economics of the process. In certain embodiments, the processes and systems of the presently disclosed subject matter generate a tertiary alkyl ether (e.g., MTBE) from an etherification reaction of isobutylene with an alcohol and accordingly do not require any separate input or source of the tertiary alkyl ether.

The process and system of generating a glycerol ether disclosed herein includes at least the following embodiments:

### Embodiment 1

A process of generating a glycerol ether, comprising: reacting isobutylene with an alcohol to obtain a tertiary alkyl ether through an etherification reaction; and generating glycerol ether from the tertiary alkyl ether and glycerol through a transetherification reaction.

### Embodiment 2

The process of Embodiment 1, wherein the glycerol ether comprises a glycerol tert-butyl ether (GTBE).

### Embodiment 3

The process of Embodiment 2, wherein the glycerol tert-butyl ether (GTBE) comprises an ether selected from di-tert-butyl glycerol ethers (di-GTBEs), tri-tert-butyl glycerol ether (tri-GTBE), or a combination comprising at least one of the foregoing.

### Embodiment 4

The process of any of Embodiments 1-3, wherein the tertiary alkyl ether is methyl tert-butyl ether.

### Embodiment 5

The process of any of Embodiments 1-4, wherein the alcohol is methanol.

### Embodiment 6

The process of any of Embodiments 1-5, wherein molecular sieves are used in the transetherification reaction.

### Embodiment 7

The process of any of Embodiments 1-6, further comprising obtaining isobutylene from a C₄ hydrocarbon mixture of alkanes and alkenes.

### Embodiment 8

The process of Embodiment 7, wherein the C₄ hydrocarbon mixture of alkanes and alkenes is C₄ raffinate-1.

### Embodiment 9

The process of Embodiment 7 or Embodiment 8, further comprising obtaining the C₄ hydrocarbon mixture of alkanes and alkenes from extracting butadiene from a C₄ hydrocarbon mixture.

### Embodiment 10

The process of any of Embodiments 1-9, wherein the transetherification reaction is catalyzed by one or more catalysts selected from liquid acids, solid acids, or a combination comprising at least one of the foregoing.

### Embodiment 11

The process of Embodiment 10, wherein one or more of the catalysts is an ion-exchange resin.

### Embodiment 12

The process of any of Embodiments 1-11, wherein the transetherification reaction regenerates an alcohol.

### Embodiment 13

The process of Embodiment 12, further comprising feeding the alcohol regenerated in the transetherification reaction into the etherification reaction.

### Embodiment 14

The process of any of Embodiments 1-13, further comprising obtaining glycerol from a biodiesel process.

### Embodiment 15

The process of Embodiment 14, wherein the biodiesel process comprises reacting fatty acids with an alcohol to generate biodiesel and glycerol.

### Embodiment 16

The process of Embodiment 15, wherein the alcohol is methanol.

### Embodiment 17

The process of any of Embodiments 1-16, further comprising feeding an alcohol generated in the transetherification reaction into a biodiesel process.

### Embodiment 18

The process of Embodiment 17, wherein the biodiesel process comprises reacting fatty acids with the alcohol to generate biodiesel and glycerol.

### Embodiment 19

The process of Embodiment 18, wherein the alcohol is methanol.

### Embodiment 20

The process of Embodiment 19, further comprising feeding the glycerol generated by the biodiesel process into the transetherification reaction.

### Embodiment 21

A system for generating a glycerol ether, comprising: a first section for generating a tertiary alkyl ether, wherein said first section comprises: at least one tertiary alkyl ether reactor coupled to an isobutylene feed line and an alcohol feed line; at least one separation unit coupled to the tertiary alkyl ether reactor; a tertiary alkyl ether outlet line coupled to the separation unit; and a tertiary alkyl ether storage tank coupled to the tertiary alkyl ether outlet line and configured to store a tertiary alkyl ether; and a second section for generating a glycerol ether from a tertiary alkyl ether and glycerol, wherein said second section comprises: a glycerol ether production unit coupled to a tertiary alkyl ether feed line, wherein the tertiary alkyl ether feed line is further coupled to the tertiary alkyl ether storage tank of the first section, and a glycerol feed line; and a glycerol ether product line configured to remove a glycerol ether from the glycerol ether production unit.

### Embodiment 22

The system of Embodiment 21, further comprising an alcohol outlet line coupled to the glycerol ether production unit of the second section and configured to remove an alcohol from the glycerol ether production unit.

### Embodiment 23

The system of Embodiment 22, further comprising a third section for generating biodiesel, wherein said third section comprises: a biodiesel production unit coupled to a fatty acid feed line and the alcohol outlet line of the second section; and a biodiesel product line configured to remove biodiesel from the biodiesel production unit.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A process of generating a glycerol ether, comprising:
reacting isobutylene with an alcohol to obtain a tertiary alkyl ether through an etherification reaction; and
generating glycerol ether from the tertiary alkyl ether and glycerol through a transetherification reaction.

2. The process of claim 1, wherein the glycerol ether comprises a glycerol tert-butyl ether (GTBE).

3. The process of claim 2, wherein the glycerol tert-butyl ether (GTBE) comprises an ether selected from di-tert-butyl glycerol ethers (di-GTBEs), tri-tert-butyl glycerol ether (tri-GTBE), or a combination comprising at least one of the foregoing.

4. The process of claim 1, wherein the tertiary alkyl ether is methyl tert-butyl ether.

5. The process of claim 1, wherein the alcohol is methanol.

6. (canceled)

7. The process of claim 1, further comprising obtaining isobutylene from a C₄ hydrocarbon mixture of alkanes and alkenes.

8. The process of claim 7, wherein the C₄ hydrocarbon mixture of alkanes and alkenes is C₄ raffinate-1.

9. The process of claim 7, further comprising obtaining the C₄ hydrocarbon mixture of alkanes and alkenes from extracting butadiene from a C₄ hydrocarbon mixture.

10. The process of claim 1, wherein the transetherification reaction is catalyzed by one or more catalysts selected from liquid acids, solid acids, or a combination comprising at least one of the foregoing.

11. The process of claim 10, wherein one or more of the catalysts is an ion-exchange resin.

12. The process of claim 1, wherein the transetherification reaction regenerates an alcohol.

13. The process of claim 12, further comprising feeding the alcohol regenerated in the transetherification reaction into the etherification reaction.

14. The process of claim 1, further comprising obtaining glycerol from a biodiesel process, wherein the biodiesel process comprises reacting fatty acids with an alcohol to generate biodiesel and glycerol.

15. (canceled)

16. (canceled)

17. The process of claim 1, further comprising feeding an alcohol generated in the transetherification reaction into a biodiesel process.

18. The process of claim 17, wherein the biodiesel process comprises reacting fatty acids with the alcohol to generate biodiesel and glycerol.

19. The process of claim 18, wherein the alcohol is methanol.

20. The process of claim 19, further comprising feeding the glycerol generated by the biodiesel process into the transetherification reaction.

21. A system for generating a glycerol ether, comprising:
a first section for generating a tertiary alkyl ether, wherein said first section comprises:
at least one tertiary alkyl ether reactor coupled to an isobutylene feed line and an alcohol feed line;
at least one separation unit coupled to the tertiary alkyl ether reactor;
a tertiary alkyl ether outlet line coupled to the separation unit; and
a tertiary alkyl ether storage tank coupled to the tertiary alkyl ether outlet line and configured to store a tertiary alkyl ether; and
a second section for generating a glycerol ether from a tertiary alkyl ether and glycerol, wherein said second section comprises:
a glycerol ether production unit coupled to a tertiary alkyl ether feed line, wherein the tertiary alkyl ether feed line is further coupled to the tertiary alkyl ether storage tank of the first section, and a glycerol feed line; and
a glycerol ether product line configured to remove a glycerol ether from the glycerol ether production unit.

22. The system of claim 21, further comprising an alcohol outlet line coupled to the glycerol ether production unit of the second section and configured to remove an alcohol from the glycerol ether production unit.

23. The system of claim 22, further comprising a third section for generating biodiesel, wherein said third section comprises:
a biodiesel production unit coupled to a fatty acid feed line and the alcohol outlet line of the second section; and
a biodiesel product line configured to remove biodiesel from the biodiesel production unit.
